(19) United States
(12) Patent Application Publication
(10) Pub. No.: US 2008/0172988 A1
(43) Pub. Date: Jul. 24, 2008

(54) Sterilization packing case and method for packing thereof

(75) Inventor: Jae Kwang Hwang, Seoul (KR)

Correspondence Address: BUCHANAN, INGERSOLL & ROONEY PC, POST OFFICE BOX 1404, ALEXANDRIA, VA, 22313-1404, US

(73) Assignee: F.S. KOREA INDUSTRIES INC., Seoul (KR)

(21) Appl. No.: 11/889,614

(22) Filed: Aug. 15, 2007

(30) Foreign Application Priority Data
Jan. 19, 2007 (KR) 10-2007-0006340

(51) Int. Cl. B65B 55/02; A61L 2/08

(52) U.S. Cl. 53/425; 422/22

(57) ABSTRACT

Disclosed is a sterilization packing case and a method for packing thereof. The present invention provides a sterilization packing case for packing finished goods under a sterile condition; and a window for sterilization provided in the sealing envelope and composed of materials. Accordingly, the sterilization packing case according to the present invention may be useful to maximize a sterilization level of the finished goods, thereby to increase preservation duration of the finished goods under the sterile condition, and improve its productivity by simplifying the sterilization process which is separately carried out for the finished goods and the packing case, as well as prevent contaminations or bacterial infections appearing in a sealing process by sterilizing finished goods such as cosmetics, medical supplies, foods and drinks through the disinfection while being sealed in the packing case, wherein the finished goods should be necessarily conserved under a sterile condition.

### TECHNICAL FIELD

The present invention relates to a sterilization packing case and a method for packing thereof, and more particularly to a sterilization packing case capable of preventing contaminations or bacterial infections appearing in a sealing process by sterilizing finished goods such as cosmetics, medical supplies, foods and drinks through the disinfection while being sealed in the packing case, wherein the finished goods should be necessarily conserved under a sterile condition, and a method for packing thereof.

### BACKGROUND ART

Generally, there have been a variety of sterilization packing methods and packing cases developed and used to provide users with cosmetics, medical supplies, foods and drinks, etc. in a sanitary condition, and simultaneously perform a sterilization treatment so as to extend their preservation duration and sustain their sterile condition for a long time.

These sterilization packing methods of the finished goods are very important in the aspects of inhibiting cosmetics, medical supplies, foods and drinks, etc. from being easily changed in quality and infected with bacteria and preventing them from causing diseases in users due to the side effects.

The conventional sterilization packing methods, as described above, are carried out by performing a sterilization process on finished goods such as cosmetics, medical supplies, foods and drinks before sealing the finished goods using various method such as steam sterilization, gas sterilization, radiation sterilization, chemical sterilization, dry heat sterilization, etc., followed by sealing a sterilization packing case.

However, the conventional sterilization packing methods have disadvantages in that sterilized finished goods may be exposed to the air and contaminated with bacteria and the like during a packing process since the finished goods are packed up in a packing case after they are sterilized, and that a sterilization process and an apparatus for sterilization are very complicated since the finished goods and the packing case are necessarily sterilized separately, and the finished goods may be also changed in quality if the sterile condition is lifted from one of the sterilization process and the apparatus for sterilization.

### DISCLOSURE

### Technical Problem

Accordingly, the present invention has been made to solve the above-mentioned problems in the prior art, and an object of the present invention is to provide a sterilization packing case and a method for packing thereof, the sterilization packing case being able to maximize a sterilization level of the finished goods, thereby to increase preservation duration of the finished goods under the sterile condition, and improve its productivity by simplifying the sterilization process which is separately carried out for the finished goods and the packing case, as well as prevent contaminations or bacterial infections appearing in a sealing process by sterilizing finished goods such as cosmetics, medical supplies, foods and drinks through the disinfection while being sealed in the packing case, wherein the finished goods should be necessarily conserved under a sterile condition.

### Technical Solution

In accordance with an aspect of the present invention, there is provided a sterilization packing case for packing finished goods under a sterile condition, the packing case including a sealing envelope having a sealing portion for hermetically sealing a packing case, the sealing portion being formed in an inlet through which the finished goods are put, and being composed of materials that do not transmit gases or beams used for disinfecting the finished goods; and a window for sterilization provided in the sealing envelope and composed of materials that transmit gases or beams used for disinfecting the finished goods.

Also, there is provided a method for packing a sterilization packing case which packs finished goods under a sterile condition, the method including: putting the finished goods into a sealing envelope through which gases or beams are transmitted, the window for sterilization being provided in some portion of the sealing envelope and the gases or beams being used for disinfecting the finished goods only through a window for sterilization; sealing the sealing envelope; and disinfecting the finished goods inside the sealing envelope by exposing the finished goods to gases or beams used for disinfecting the finished goods through a window for sterilization.

### BRIEF DESCRIPTION OF THE DRAWINGS

The foregoing and other objects, features and advantages of the present invention will become more apparent from the following detailed description when taken in conjunction with the accompanying drawings.

FIG. 1 is a perspective view showing a sterilization packing case according to the present invention.

FIG. 2 is a flowchart illustrating a sterilization packing method according to the present invention.

### DESCRIPTIONS OF MAJOR PARTS IN THE DRAWINGS

11: sealing envelope
12: window for sterilization
13: sealing portion
14: sterilization-confirming tag

### BEST MODE

Hereinafter, preferred embodiments of the present invention will be described with reference to the accompanying drawings, so that the present invention may be easily put into practice by those skilled in the art.

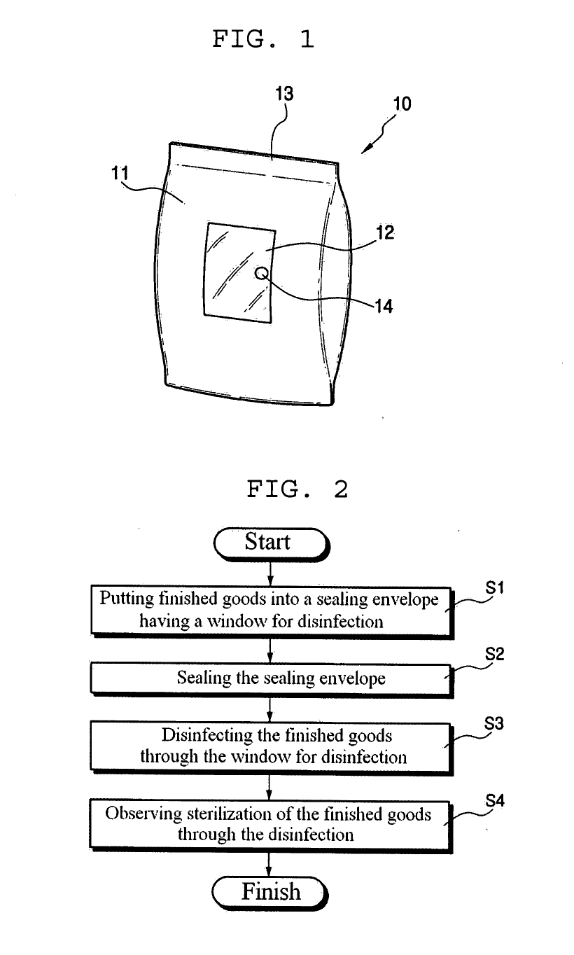

FIG. 1 is a perspective view showing a sterilization packing case according to the present invention. As shown in FIG. 1, the sterilization packing case 10 according to the present invention includes a sealing envelope 11 composed of materials that do not transmit gases or beams used for disinfecting the finished goods such as cosmetics, medical supplies, foods and drinks; and a window 12 for sterilization provided in the sealing envelope 11 and composed of materials that transmit gases or beams used for disinfecting the finished goods.

The sealing envelope 11 is provided with an inlet (not shown) for putting finished goods into one side, for example an upper portion, of the sealing envelope 11 and a sealing portion 13 for hermetically sealing a packing case formed in the inlet.

The sealing portion 13 may be sealed with an adhesive or attached using the heat, and a zipper and the like may also be used to open and shut a packing case in a repeated manner unlike the single sealing process.

The sealing envelope 11 is composed of various materials not to transmit gases or beams used for disinfecting the finished goods, and preferably composed of thin silver foils which are made of a single- or multiple-fold silver, or silver-colored materials.

The window for sterilization 12 is provided in one side of the sealing envelope 11, and may be composed of various materials, preferably synthetic resin materials such as low density polyethylene (LDPE), high low density polyethylene (HDPE), polypropylene (PP), polyvinyl chloride (PVC), polystyrene (PS), and the like, to transmit gases or beams used for disinfecting the finished goods.

The window for sterilization 12 may be composed of transparent materials, for example transparent synthetic resin materials, preferably linear low density polyethylene (LLDPE), so that the finished goods included inside a packing case can be observed with the naked eye.

The window for sterilization 12 may have a sterilization-confirming tag 14 installed in an inner side thereof, and the sterilization-confirming tag 14 displays whether or not the finished goods are disinfected through the changes in colors when the sterilization is completed through the disinfection, for example when the sterilization-confirming tag 14 is changed from a brown color to a green color.

Meanwhile, the gases or beams used for disinfecting finished goods are preferably ethylene oxide gas (EO gas) or gamma ray.

FIG. 2 is a flowchart illustrating a sterilization packing method according to the present invention. As shown in FIG. 2, the sterilization packing method according to the present invention includes steps of putting finished goods into a sealing envelope having a window for sterilization (S1); sealing a sealing envelope (S2); and disinfecting the finished goods inside the sealing envelope through the window for sterilization (S3).

In the step of putting finished goods into a sealing envelope having a window for sterilization (S1), the finished goods such as cosmetics, medical supplies, foods and drinks are passed through an inlet into the sealing envelope 11 (not shown in FIG. 1) through which gases or beams are transmitted, the sealing envelope 11 being composed of materials such as silver foils and the gases or beams being used for disinfecting the finished goods only through the window 12 for sterilization (not shown in FIG. 1) provided in some portion of the sealing envelope.

In the step of sealing a sealing envelope (S2), the sealing portion 13 is sealed with an adhesive or attached using the heat, or sealed with a means such as a zipper and the like, wherein the sealing portion 13 is provided in the inlet of the sealing envelope 11, the inside of which the finished goods are put into.

In the step of disinfecting finished goods through a window for sterilization (S3), the finished goods inside the sealing envelope 11 are disinfected by exposing gases or beams which are used for disinfecting finished goods through the window for sterilization 12. For example, the finished goods are disinfected by penetrating an ethylene oxide gas (EO gas) into the window for sterilization 12 of the hermetically sealed sealing envelope 11 or irradiating a gamma ray, and therefore the finished goods are kept under a sterile condition. At this time, the disinfection time and temperature may be varied according to the kind of the finished goods, and the disinfection step is preferably carried out at a temperature range from 10 to 60° C. for approximately 30 minutes to 10 hours.

Meanwhile, after the step of disinfecting finished goods through a window for sterilization (S3), it is exactly determined whether or not the finished goods is sterilized, by performing a step of observing sterilization of the finished goods through the disinfection (S4).

In the observing sterilization of the finished goods through the disinfection (S4), the sterilization of the finished goods is determined through the change in colors of the above-mentioned sterilization-confirming tag 14 which is provided inside the window for sterilization 12 composed of a transparent material, for example a PVC material.

As described above, the sterilization packing case according to the preferred embodiments of the present invention has advantages in that the packing case maximizes a sterilization level of the finished goods, thereby to increase preservation duration of the finished goods under the sterile condition, and improves its productivity by simplifying the sterilization process which is separately carried out for the finished goods and the packing case, as well as prevents contaminations or bacterial infections appearing in a sealing process by sterilizing finished goods such as cosmetics, medical supplies, foods and drinks through the disinfection while being sealed in the packing case, wherein the finished goods should be necessarily conserved under a sterile condition.

As described above, the sterilization packing case according to the present invention and the method for packing thereof may be useful to maximize a sterilization level of the finished goods, thereby to increase preservation duration of the finished goods under the sterile condition, and improve its productivity by simplifying the sterilization process which is separately carried out for the finished goods and the packing case, as well as prevent contaminations or bacterial infections appearing in a sealing process by sterilizing finished goods such as cosmetics, medical supplies, foods and drinks through the disinfection while being sealed in the packing case, wherein the finished goods should be necessarily conserved under a sterile condition.

As described above, the sterilization packing case according to the present invention and the method for packing thereof have been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description, as defined in the appended claims.

1. A sterilization packing case for packing finished goods under a sterile condition, the packing case comprising:
a sealing envelope having a sealing portion for hermetically sealing a packing case, the sealing portion being formed in an inlet through which the finished goods are put, and being composed of materials that do not transmit gases or beams used for disinfecting the finished goods; and
a window for sterilization provided in the sealing envelope and composed of materials that transmit gases or beams used for disinfecting the finished goods.

2. The sterilization packing case according to claim 1, wherein the gases or beams used for disinfecting the finished goods is ethylene oxide gas (EO gas) or gamma ray.

3. The sterilization packing case according to claim 1, wherein the sealing envelope is composed of silver foil material.

4. The sterilization packing case according to claim 1, wherein the window for sterilization is composed of transparent PVC material.

5. The sterilization packing case according to claim 4, further comprising a sterilization-confirming tag installed inside the window for sterilization and indicating from the outside whether or not the finished goods are sterilized.

6. A method for packing a sterilization packing case which packs finished goods under a sterile condition, the method comprising:
putting the finished goods into a sealing envelope through which gases or beams are transmitted, the window for sterilization being provided in some portion of the sealing envelope and the gases or beams being used for disinfecting the finished goods only through a window for sterilization;
sealing the sealing envelope; and
disinfecting the finished goods inside the sealing envelope by exposing the finished goods to gases or beams used for disinfecting the finished goods through a window for sterilization.

7. The method according to claim 6, wherein, in the step of disinfecting the finished goods inside the sealing envelope, the gases or beams used for disinfecting the finished goods is ethylene oxide gas (EO gas) or gamma ray.

8. The method according to claim 6, after the step of disinfecting the finished goods inside the sealing envelope, further comprising a step of observing through a sterilization-confirming tag whether or not the finished goods is sterilized, the sterilization-confirming tag being provided inside the transparent window for sterilization.
